(19) United States
(12) Patent Application Publication
(10) Pub. No.: US 2021/0330836 A1
(43) Pub. Date: Oct. 28, 2021

(54) LED LIGHT FIXTURE WITH GERMICIDAL EFFECT

(71) Applicant: Manaflex, LLC, Waikoloa, HI (US)

(72) Inventor: Robert Clinton Lane, Waikoloa, HI (US)

(21) Appl. No.: 17/240,791

(22) Filed: Apr. 26, 2021

Related U.S. Application Data
(60) Provisional application No. 63/015,564, filed on Apr. 25, 2020.
(60) Provisional application No. 63/016,971, filed on Apr. 28, 2020.

(51) Int. Cl. A61L 2/10; A61L 2/18; A61L 2/24; A61L 9/14; A61L 9/20; F21V 29/58; F21V 29/67; F21S 4/28

(52) U.S. Cl. CPC A61L 2/10; A61L 2/18; A61L 2/24; A61L 9/14; A61L 2/0047; F21V 29/59; F21V 29/67; F21S 4/28; A61L 9/20

(57) ABSTRACT

A LED light fixture having a mixture of red and white LEDs, as well as any combinations of UVA, UVB, UVC LEDs. The ratio of outputs from these LEDs may be adjusted to achieve a desired germicidal effect. The light fixture is liquid-cooled using a cooling tube that directly cools the power supply and the LED flexible printed circuit.

### CROSS-REFERENCE TO RELATED APPLICATIONS

This application claims priority to U.S. provisional patent application No. 63/015,564, filed on Apr. 25, 2020, and U.S. provisional patent application No. 63/016,971, filed on Apr. 28, 2020, both of which are now pending and are hereby incorporated by reference in their entireties.

Although incorporated by reference in its entirety, no arguments or disclaimers made in the parent application apply to this application.

### FIELD OF THE DISCLOSURE

The present disclosure relates to a lighting system for many uses such as medical, therapeutic, sanitation, and agricultural.

### BACKGROUND OF THE DISCLOSURE

Currently there are no effective ways to volumetrically sterilize surfaces and aerosols in densely populated large indoor public/private areas for viruses, bacteria, and mold in a way that is not disruptive to the economic activity that occurs in these locations.

Also, there is currently no effective ways to sterilize a live plant indoor to prevent pests/molds/viruses/bacteria from afflicting them.

There is a need to efficiently and/or effectively provide lighting and misting/watering of a plant.

Currently known uses of LED (light-emitting diode) lightings indoors or in greenhouses experience extra heat generated by the LED lightings. These known LED lights use direct cooling with forced or passive air cooling which dumps the heat within the greenhouses or buildings. As a result, additional cooling (e.g., by a HVAC system) is needed to compensate extra LED-generated heat.

There is a need to resolve current issues in LED light inefficiencies due to LED-generated heat.

There is a need to improve energy efficiency and resolve cooling issues inside of a greenhouse.

The disclosed embodiments may seek to satisfy one or more of the above-mentioned needs. Although the present embodiments may obviate one or more of the above-mentioned needs, some aspects of the embodiments might not necessarily obviate them.

### SUMMARY OF THE DISCLOSURE

Currently there are no cost-effective solutions to commercially sterilize large high bays or warehouses. In a general implementation, the disclosure provides a LED (light-emitting diode) light fixture having a LED flexible printed circuit, wherein the LED flexible printed circuit has a first array of high-power white LEDs to produce a first output of light.

In another aspect combinable with the general implementation, the LED flexible printed circuit has a second array of red LEDs to produce a second output of light.

In another aspect combinable with the general implementation, the LED flexible printed circuit has a third array of UV LEDs to produce a second output of light.

In another aspect combinable with the general implementation, the third array of UV LEDs includes at least one of UVA LEDs, UVB LEDs, and UVC LEDs.

In another aspect combinable with the general implementation, the light fixture can have dimming function for a user to separately dim any of these lights and control the ratio of UVA, UVB, UVC, red, and white wavelengths to create modes depending on the need/function/use.

The inventor has discovered that to implement UV safely it must be paired with enough visible white light to ensure that humans do not stare at the light too long which may cause cataracts. The sun emits a UV index of 0 to 20 depending on latitude. It is hypothesized that the seasonal flu is a function mainly of UV index. Because UVC, UVB, and UVA have germicidal properties, the contemplated light fixture can have the ability to control the emitted UV light index from 0 to higher than 20 depending on the need/function/use.

In one contemplated system, the light fixture or a network of such light fixture can be remotely controlled via an TOT network or other types of networks. If a pandemic or a local infection event breaks out, one could activate and adjust a desired number of lights/regions to an ideal UV index to kill microbes on surfaces within a short amount of time, even seconds, or within a desired time frame. At desired UV index such lights can also eliminate or drastically decrease aerosol microbes. Further, the contemplated system can be programmed based on feedback from the MH or CDC depending on the severity of the outbreak in each city and town.

In one example, the contemplated system can be manually/automatically/remotely/selectively turned on during certain peak dangerous times or dates and then when the threat is eliminated return to safer levels of UV index. This principal could be used in all artificial lighting in every known uses and locales. This idea could also help with indoor farming of plants because the contemplated light fixture could inactivate germs on plant surfaces and/or soil.

In one embodiment, approximately 5% of the total light output of the light fixture is UVA and UVB. Of this portion, 95% can be UVA and 5% can be UVB. Other ratios of UVA and UVB are also specifically contemplated. A function can be provided so the user may selectively dim the UVA or UVB depending on the needs at the time.

In some embodiments, the total light output of the contemplated LED light fixture is defined as the luminous power flux. In one particular embodiment, the white lights will have 95% of the luminous power flux and about 5% of the light will be the UVA spectrum. In some embodiments, the white lights can have between 90-95% of the luminous power flux. In some embodiments, the white lights can have between 85-97% of the luminous power flux. In some embodiments, the white lights can have between 92-97% of the luminous power flux.

In some embodiments, the combination of red and white lights can have between 90-95% of the luminous power flux. In some embodiments, the combination of red and white lights can have between 85-97% of the luminous power flux. In some embodiments, the combination of red and white lights can have between 92-97% of the luminous power flux.

In some embodiments, the output of UV LED is between 2% and 8% of a total light output. In some embodiments, the output of UV LED is about 2% and 10% of the total light output. In some embodiments, the output of UV LED is about 3% and 6% of the total light output. In some embodiments, the output of UV LED is about 5% of the total light output.

It is further contemplated that this lighting system could be direct liquid-cooled or air cooled. If liquid-cooled then any known suitable liquid can be used such as water, coolant, fertilizing liquid; liquid nutrient, and disinfectant. Some contemplated embodiments can also dispense the liquid in the form of a spray, droplets, or fine mist, on to the plants or to control humidity level in the environment. Furthermore, the activation of the misting can create a highly efficient phase-change cooling effect.

Among the many possible LED light fixture configurations, the design of the lighting system can be highly efficient. Contemplated LED arrays can connect directly into power supplies with minimum metal connection/wiring for conducting electrons. Also, power supplies can function as a heat sink or heat exchanger to the LED array. This can reduce cost for heat sink, enclosure, and metal for conducting electrons.

While this specification contains many specific implementation details and examples, these should not be construed as limitations on the scope of any inventions or of what may be claimed, but rather as descriptions of features specific to particular implementations of particular embodiments.

Certain features that are described in this specification in the context of separate implementations can also be implemented in combination in a single implementation. Conversely, various features that are described in the context of a single implementation can also be implemented in multiple implementations separately or in any suitable subcombination.

The details of one or more implementations of the subject matter described in this disclosure are set forth in the accompanying drawings and the detailed description below. Other features, aspects, and advantages of the subject matter will become apparent from the description, the drawings, and the claims.

### BRIEF DESCRIPTION OF THE DRAWINGS

It should be noted that the drawing figures may be in simplified form and might not be to precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a perspective view of one embodiment of the contemplated LED light fixture, according to one aspect of the disclosure.

FIG. 2 is a perspective view of the embodiment of the contemplated LED light fixture of FIG. 1 turned to its side showing the bottom side of the LED light fixture, according to one aspect of the disclosure.

FIG. 3 is a close-up of the circled region marked A in FIG. 2, according to one aspect of the disclosure.

FIG. 4 is a top view of the embodiment of the contemplated LED light fixture of FIG. 1, according to one aspect of the disclosure.

FIG. 5 is a side view of the embodiment of the contemplated LED light fixture of FIG. 1, according to one aspect of the disclosure.

FIG. 6 is a bottom view of the embodiment of the contemplated LED light fixture of FIG. 1, according to one aspect of the disclosure.

FIG. 7 is a close-up of the section marked B in FIG. 6, according to one aspect of the disclosure.

FIG. 8 is an exploded view of the embodiment of the contemplated LED light fixture of FIG. 1, according to one aspect of the disclosure.

FIG. 9 is a close-up of the circled region marked C in FIG. 8, according to one aspect of the disclosure.

FIG. 10 is a close-up of the circled region marked D in FIG. 8, according to one aspect of the disclosure.

FIG. 11 is a bottom perspective view of an embodiment of the cooling tube assembly sandwiched by power supplies and LED flexible printed circuits, according to one aspect of the disclosure.

FIG. 12 is a close-up of the circled region marked E in FIG. 11, according to one aspect of the disclosure.

FIG. 13 is a top perspective view of an embodiment of the cooling tube assembly sandwiched by power supplies and LED flexible printed circuits, according to one aspect of the disclosure.

FIG. 14 is a close-up of the circled region marked F in FIG. 13, according to one aspect of the disclosure.

FIG. 15 is a close-up of the circled region marked G in FIG. 13, according to one aspect of the disclosure.

FIG. 16 is a close-up of the circled region marked H in FIG. 13, according to one aspect of the disclosure.

FIG. 17 is a perspective view of a solid-to-air heat exchanger with a series of electric fans attached, according to one aspect of the disclosure.

FIG. 18 is a perspective view of the solid-to-air heat exchanger with a series of electric fans of FIG. 17, wrapped with a LED flexible printed circuit, according to one aspect of the disclosure.

FIG. 19 is a simplified illustration of an embodiment of air-cooled LED light fixture using a solid-to-air heat exchanger with electric fans and having power supplies and LED flexible printed circuits attached thereto, according to one aspect of the disclosure.

FIG. 20 is an illustration of an embodiment of liquid-cooled LED light fixture using a remote radiator located outside, according to one aspect of the disclosure.

FIG. 21 is an illustration of an embodiment of liquid-cooled LED light fixture using a radiator located indoor, according to one aspect of the disclosure.

FIG. 22 is an illustration of an embodiment of liquid-cooled LED light fixture using a radiator located indoor and a radiator located outdoor, according to one aspect of the disclosure.

The following call-out list of elements in the drawing can be a useful guide when referencing the elements of the drawing figures:
4 Indoor
5 Outdoor
100 LED Light Fixture
102 Housing
104 On/Off Switch
108 Power Connector
120 LED Flexible Printed Circuit
121 High-power white LED
122 Red LED
124 UV LED
125 Pins
130 Power Supply
131 Contacts
132 Power Board
140 Buss Flexible Printed Circuit
150 Cooling Tube Assembly
152 Solenoid Valve
154 Conduit
156 Misting Nozzle
157 Valve
158 Radiator
159 Tube
160 Heat Exchanger
162 Fan

### DETAILED DESCRIPTION OF THE EMBODIMENTS

The different aspects of the various embodiments can now be better understood by turning to the following detailed description of the embodiments, which are presented as illustrated examples of the embodiments as defined in the claims. It is expressly understood that the embodiments as defined by the claims may be broader than the illustrated embodiments described below.

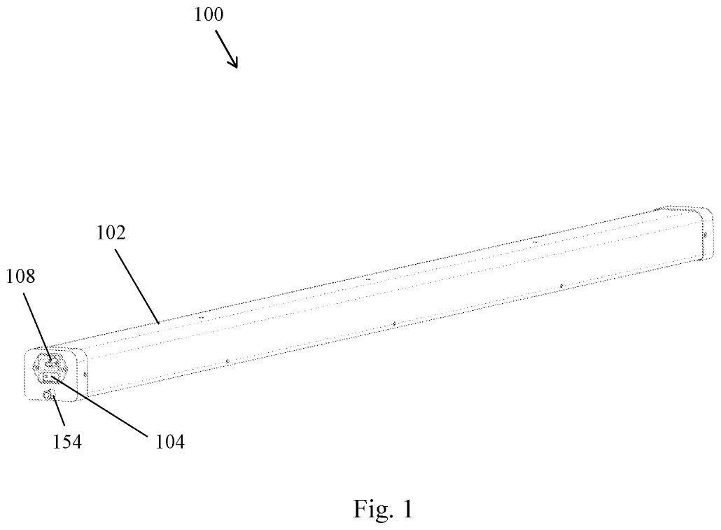

Referring now to FIG. 1, the contemplated LED light fixture 100 can have a generally elongated configuration, but the disclosure is not limited thereto. The general profile can be of a cylinder. In some embodiments, this cylinder may have a square cross-sectional shape as shown in FIG. 1, but the disclosure is not limited thereto. In some embodiments, this cylinder may have a rectangular cross-sectional shape, but the disclosure is not limited thereto. In some embodiments, this cylinder may have a round cross-sectional shape, but the disclosure is not limited thereto. In some embodiments, this cylinder may have an oval cross-sectional shape, but the disclosure is not limited thereto. In some embodiments, this cylinder may have a polygonal cross-sectional shape, but the disclosure is not limited thereto. In some embodiments, this cylinder may have any other cross-sectional shapes, but the disclosure is not limited thereto.

In one aspect of the embodiments, there can be a housing 102 to enclose all or some of the components of the contemplated light fixture 100. The various contemplated components will be described in more details below.

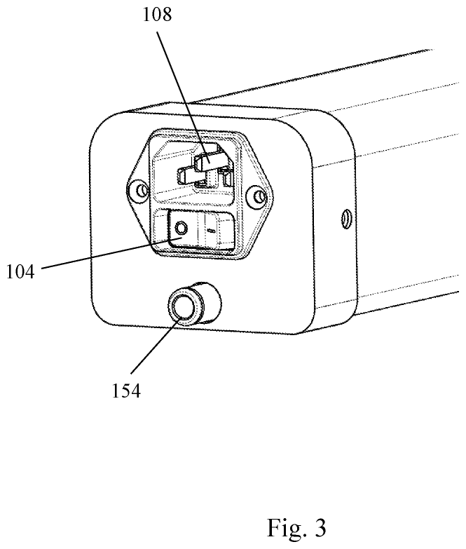

There can be a power on/off switch 104 (see also FIG. 3) disposed on the light fixture 100. In the embodiment contemplated and illustrated in FIG. 1, the power on/off switch 104 is disposed on a terminal end of the light fixture 100, but the disclosure is not limited thereto.

There can be a power connector 108 (see also FIG. 3) disposed on the light fixture 100 to connect to a power source. In the embodiment contemplated and illustrated in FIG. 1, the power connector 108 is disposed on a terminal end of the light fixture 100, but the disclosure is not limited thereto.

Some of the contemplated embodiments of the present disclosure may include a conduit 154 (see also FIG. 3) disposed on the light fixture 100. In the embodiment illustrated in FIG. 1, the conduit is disposed on a terminal end of the light fixture 100. The function and purpose of the conduit 154 will be described in more details later.

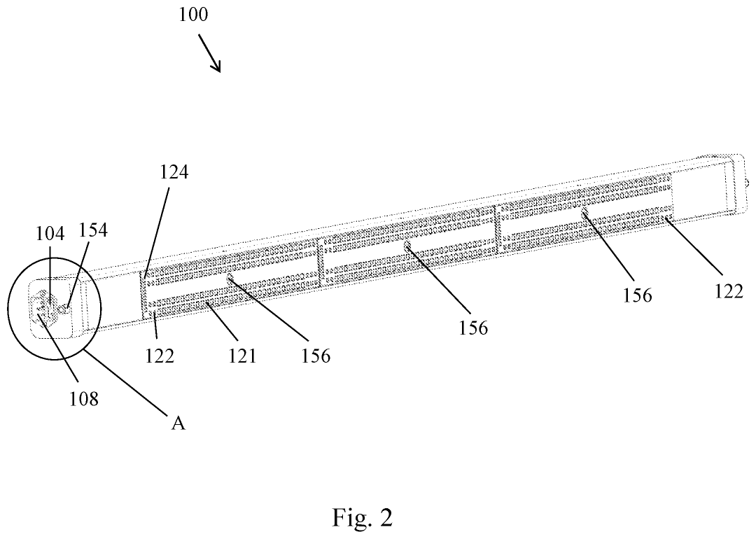

Referring now to FIG. 2, the light fixture 100 in FIG. 2 is flipped on its side to illustrate the underside of the contemplated light fixture 100. The contemplated light fixture 100 features an array of LEDs 121, 122, 124 disposed on the underside of the light fixture 100. This array of LEDs 121, 122, 124 will be described in more details later.

In some particularly contemplated embodiments, there can be misting nozzles 156 disposed on the underside of the light fixture 100 such as those shown in FIG. 2. The misting nozzles 156 can selectively or automatically spray water mist (or any other desired liquid mist) to an area under the light fixture 100. The details of the misting nozzles 156 will be described in more details below.

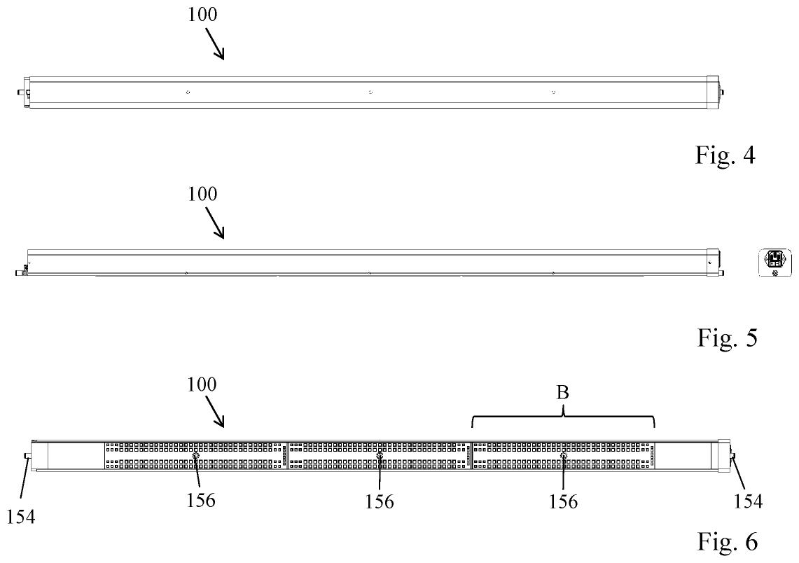

FIG. 4 illustrates a contemplated top view of the LED light fixture 100, accordingly to one aspect of the disclosure. It can be generally straight and elongated. Its length, however, can be customized based on the needs of the area to be sanitized. It is also particular contemplated that the LED light fixture 100 does not have to be straight and can be customized based on the needs of the area to be sanitized.

FIG. 5 illustrates a contemplated side view of the LED light fixture 100, accordingly to one aspect of the disclosure. The side can be generally horizontally flat and elongated. Its length, however, as discussed above, can be customized based on the needs of the area to be sanitized. It is also particular contemplated that the LED light fixture 100 does not have to be horizontally flat and can be customized based on the needs of the area to be sanitized.

FIG. 6 illustrates a contemplated side view of the LED light fixture 100, accordingly to one aspect of the disclosure. The side can be generally horizontally flat and elongated. Its length, however, as discussed above, can be customized based on the needs of the area to be sanitized. It is also particular contemplated that the LED light fixture 100 may not be horizontally flat and can have a customized configuration based on the needs of the area to be sanitized.

### LEDs

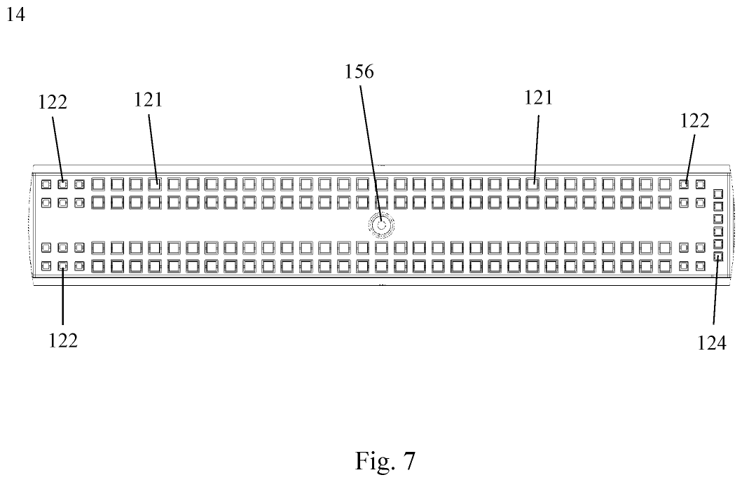

Referring now to FIG. 7 which is a close-up view of section B indicated on FIG. 6. Within section B, there can be a high density of LEDs 121, 122, 124 on a single LED flexible printed circuit 120. This single piece of LED flexible printed circuit 120 may be representative of the remaining portions of the LED light fixture 100 where LEDs are present. The contemplated LED flexible printed circuit 120 can have an array of LEDs including high-power white LEDs 121, the red LED 122, and UV LEDs 124. In some embodiments, the UV LEDs 124 may include any numbers of UVA LEDS, UVB LEDs and UVC LEDs. Alternatively, UV LEDS 124 includes only one of UVA LEDS, UVB LEDS, and UVC LEDS.

Optionally, the LED lighting fixture 100 can have one or more switching integrated circuits to pulse the white and red LEDs from 0 to maximum power separate from the switching transistor that pulses the LEDs for the UV array (UVA, UVB, UVC).

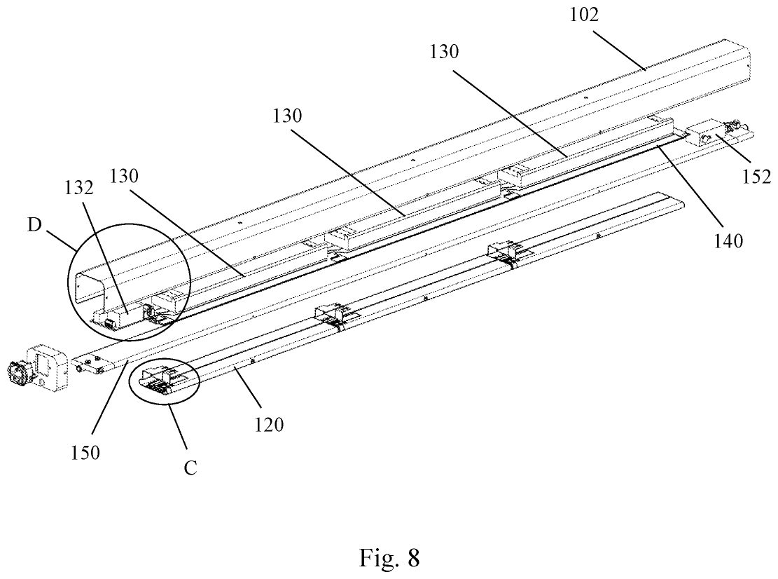

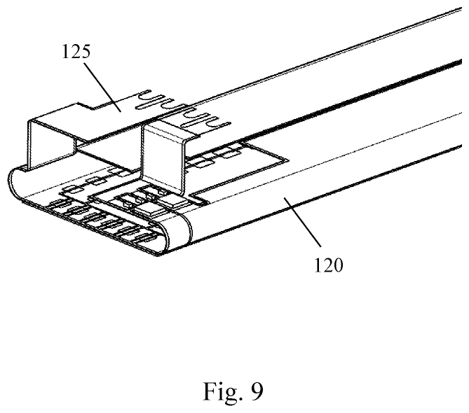

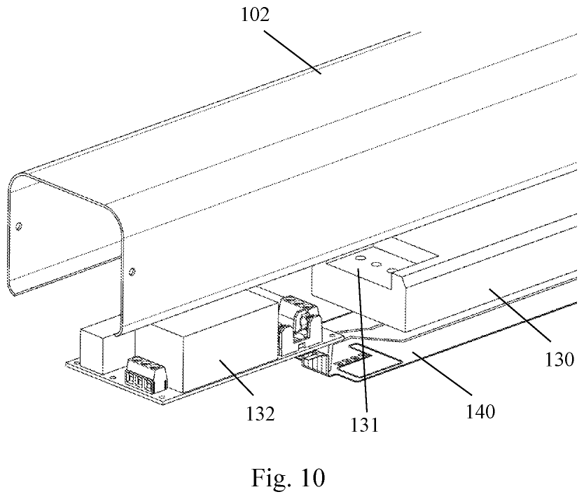

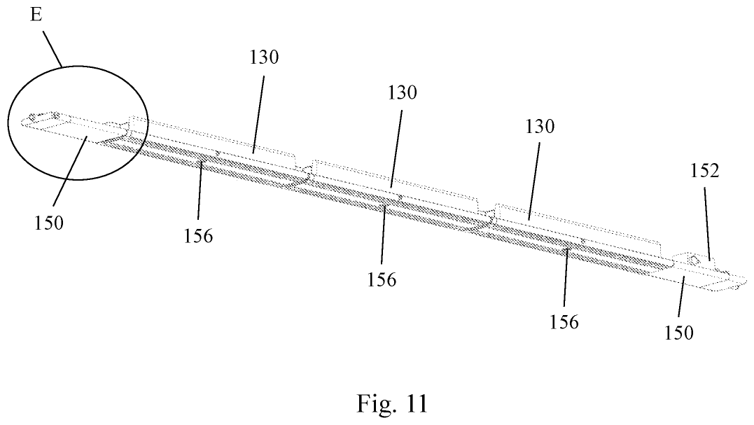

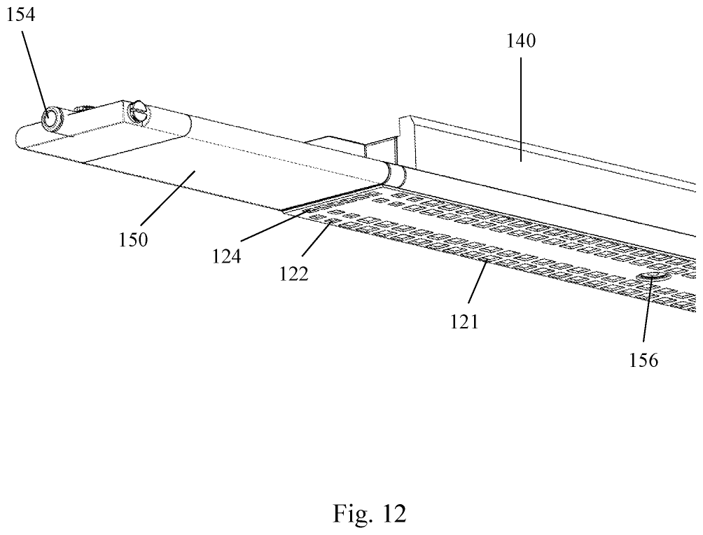

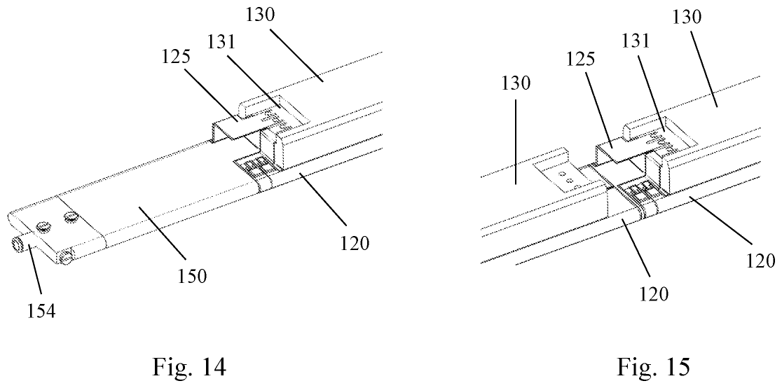

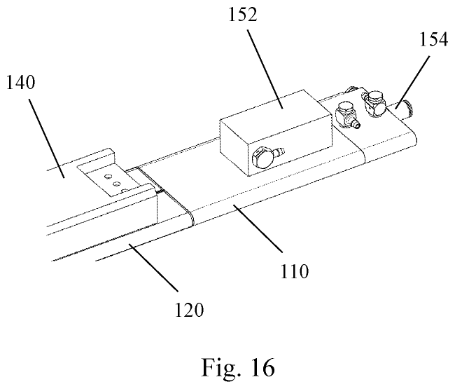

The exploded view of FIG. 8 illustrates the spatial relationships between the LED flexible printed circuit 120 and other components of the LED light fixture 100. Here, the housing 102 can have a cross-sectional U shape forming a trough to enclose some or all the inner components. The bottommost component can be three of the same LED flexible printed circuits 120 arranged end-to-end. In some embodiments, these three LED flexible printed circuits 120 may not be electrically connected end-to-end (see FIG. 15). The close-up view in FIG. 9 shows the front end of one LED flexible printed circuit 120. The LED flexible printed circuit 120 can form a cross-sectional U shape sleeve. In the embodiment shown in FIG. 9, a cross-sectional O shaped sleeve is formed having an open slit disposed lengthwise on the top side of the O shaped sleeve. The sleeve creates a through channel such that a cooling tube assembly 150 may be inserted therethrough (see FIGS. 11-16). In other words, a contemplated LED flexible printed circuit 120 can wrap around the cooling tube assembly 150 and be electrically connected to a corresponding power supply 130 via pins 125 (see FIGS. 9, 14) and contacts 131 (see FIGS. 10, 14).

In some embodiment, besides the pins 125 and the contacts 131, there are no wires or cable necessarily present to connect the LEDs 121, 122, 124 to the power supplies 130.

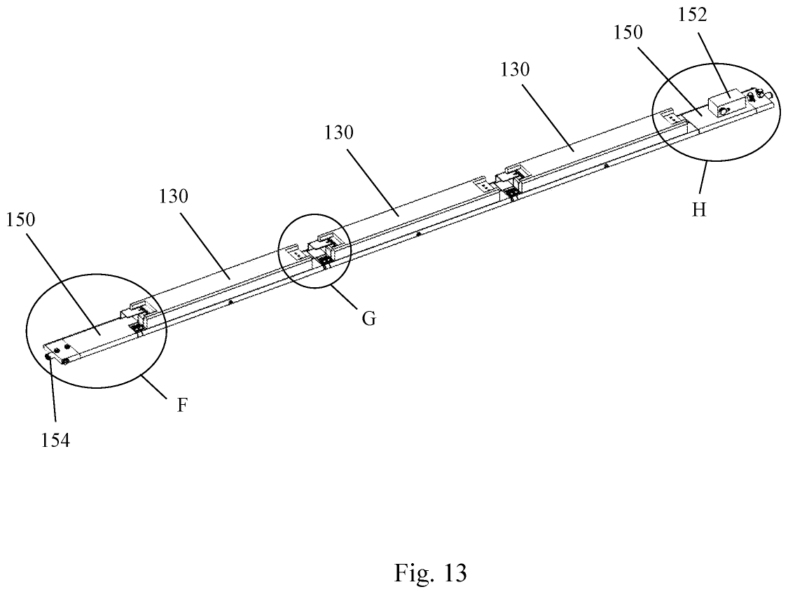

FIG. 14 is a close-up view of the circle region F of FIG. 13. Here, the first LED flexible printed circuit 120 is shown to have wrapped around the cooling tube assembly 150. The terminal portion of the cooling tube assembly 150 can remain exposed.

Returning now to FIG. 8, each of the three LED flexible printed circuits 120 can individually wrap around the same single cooling tube assembly 150. In other words, this embodiment provides only one single cooling tube assembly 150 to which each of the three LED flexible printed circuits 120 can individually wrap around. Another aspect of some embodiments provides that each of the three LED flexible printed circuits 120 can electrically connect to a corresponding power supply 130. Each corresponding power supply 130 can be disposed above the cooling tube assembly 150 and can have a relatively flat bottom surface to make maximum surface (directly or indirectly) contact with the flat top surface of the cooling tube assembly 150.

In one aspect of the embodiments discussed above, the combination of one LED flexible printed circuit 120 with one power supply 130 can create a LED module. This modularized design can allow for quick assembly, low-cost production, and easily customizable lengths by simply adding these LED modules (each of an equal length) together to achieve the desired length of LED light fixture. In addition, there can be cooling tube assemblies 150 of various desired lengths to which these LED modules can be attached to.

FIG. 15 is a close-up view of the circled region G of FIG. 13. The interface between two adjacent LED modules is illustrated here. In one embodiment, the LED flexible printed circuit 120 on the left is not contemplated to directly connect to the LED flexible printed circuit 102 on the right. The LED flexible printed circuit 120 on the left can be directly connected to the power supply 130 directly above it. Similarly, the LED flexible printed circuit 120 on the right can be directly connected to the power supply 130 directly above it.

### Liquid Cooling and Misting

It is important to appreciate that one aspect of the current disclosure may include achieving high power density at lower cost by utilizing liquid cooling. This allows much smaller units of light fixtures to be installed and at lower operating costs. One single array of LEDs can be twice more powerful than a typical greenhouse grow light such as the GAVITA PRO, and at half the operating cost. In some embodiment, the contemplated cooling tubing assembly allows for a very low thermal resistance from the LED to the liquid thereby enables the highest density of LED and thus reduce cost of the non-led components.

In these particularly contemplated embodiments, water or other liquid can be used to cool the power supply 130 and the LED flexible printed circuit 120. Referring to FIGS. 8, 11, 12-16, as discussed above, some embodiments provides that the LED flexible printed circuit 120 can wrap around the cooling tube assembly 150, thereby making direct physical contact with at least the bottom surface of the cooling tube assembly 150. In some embodiments, the LED flexible printed circuit 120 is substantially entirely wrapped around the cooling tube assembly 150 such that its entire surface on one side makes physical contact with the cooling tube assembly 150.

In FIG. 11, the cooling tube assembly 150 is shown to have three LED flexible printed circuits 120 wrapped around it, and three power supplies 130 are shown disposed above the one cooling tube assembly 150. Here, the cooling tube assembly 150 makes direct and/or indirect physical contact with at the LED flexible printed circuits 120 and the power suppliers 130, thereby cooling them.

In one contemplated embodiment, the cooling tube assembly 150 has an interior space that can be filled with a liquid such as water. When the water heats up, the water can be transported to another location via conduit 154 (see FIGS. 1-3). A fresh supply of water can be introduced through another conduct 154 (see FIG. 6) disposed on the opposite end of the LED light fixture 100.

When the water is transported to another location, the water may be ejected or recycled by cooling it at another location with a radiator/fan system.

Alternatively, or optionally, some embodiments of the LED light fixture 100 can have misting nozzles 156 disposed on the outside of the LED light fixture 100. In some embodiment, the misting nozzles 156 are disposed on the bottom side of the LED light fixture 100. In some embodiment, the misting nozzles 156 are disposed on the bottom side of the cooling tube assembly 150 and can expose through appropriated placed through holes created on the LED flexible printed circuit 120. For example, FIG. 7 shows a LED flexible printed circuit having a circular opening through which a misting nozzle 156 of the cooling tube assembly 150 is exposed. The act of misting can cause a cooling effect for the LED light fixture 100 which lessens the burden and need of using an air heat exchanger.

Alternatively, or optionally, the misting nozzles 156 can be used to spray disinfecting aerosols. In some embodiments, the disinfecting liquid can act as a cooling liquid.

Although the nozzle 156 may be described in this disclosure as a “misting” nozzle 156, its dispensing method can include all types of spraying patterns, flow rate, and size of liquid droplets, including but not limited to fine misting and dripping.

Referring now to FIGS. 11, 13, and 16, the contemplated cooling tube assembly 150 can have a solenoid valve 152 disposed on one end of the cooling tube assembly 150. The contemplated solenoid valve 152 can control the opening and closing of misting nozzles 156.

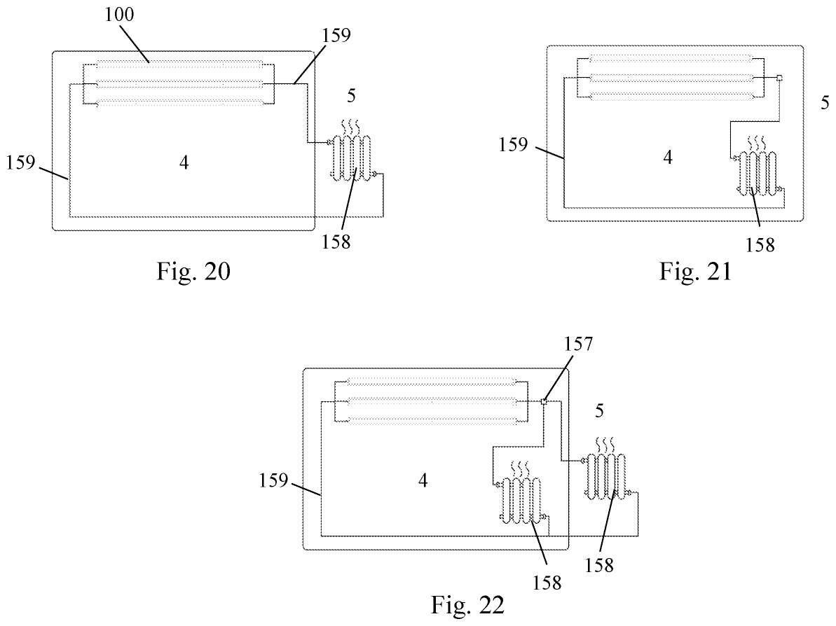

Optionally or additionally, the heated liquid can be transported away from the LED light fixture. Referring now to FIG. 20, in some embodiments, there can be a radiator 158 or a cooling tank located in an outdoor area 5 and is fluidly connected to the cooling tube assembly via a tube 159. This arrangement can keep as much heat away from the indoor environment 4 as possible. In some embodiments (see FIG. 21), there can be a radiator 158 or a cooling tank located in an indoor area 4 and is fluidly connected to the cooling tube assembly via a tube 159. This is preferred for climates where the user may want to recycle or retain the heat to keep certain part of the indoor space 4 warm, for example. In yet other embodiments (see FIG. 22), the can be both an indoor radiator 158 and an outdoor radiator 158 connected to the LED light fixture via a tube 159. A central processing unit can use appropriate sensors, thermostats, valves 157 to determine and control when the heated water should be transported to the indoor radiator 158 and when to the outdoor radiator 158.

### Air Cooling

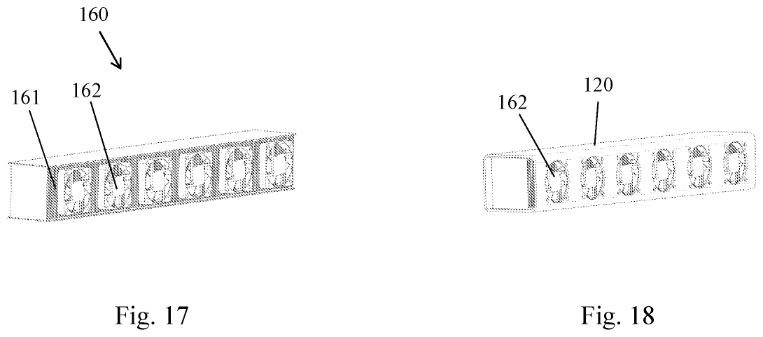

Alternatively, or optionally, the contemplated light fixture can have a heat exchanger 160 such as the one shown in FIG. 17. In FIG. 17, there can be a high-density fin structure 161 in this solid-to-air heat exchanger 160. In some embodiments, there can be at least one fan 162 coupled to the high-density fin structure 161. Contemplated fans 162 can be placed on both lateral sides of the high-density fin structure 161 to force the flow of air from one side to the other.

Referring now to FIG. 18, the contemplated LED light fixture 100 can have its LED flexible printed circuit 120 coupled to the heat exchanger 160 via an adhesive or any known thermal interface material (TIM). Here, the LED flexible printed circuit 120 can be sufficiently wide to entirely wrap around the heat exchanger 160. The LED flexible printed circuit 120 can have through openings to expose the series of fans 162 disposed on both lateral sides of the heat exchanger 160, thereby allowing unobstructed flow of air.

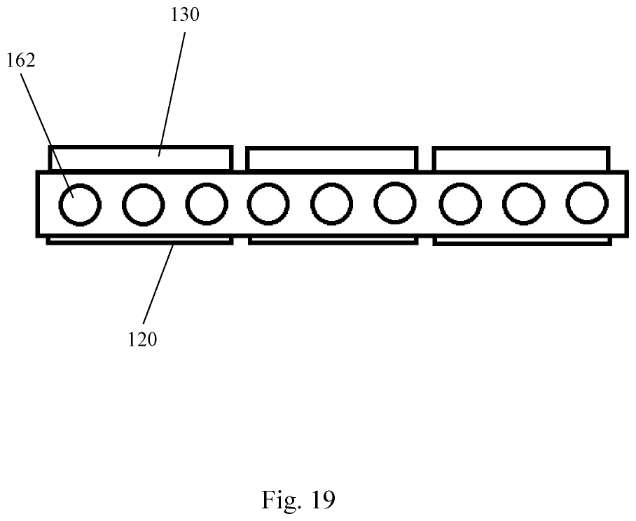

Referring now to FIG. 19, another contemplated embodiment is shown where at least one power supply 130 may be disposed above the heat exchanger 160 and at least one LED flexible printed circuits 120 can be disposed below the heat exchanger 160 via an adhesive, a thermal interface material, or other known means.

Alternatively, or optionally, the contemplated light fixture 100 can utilize a large natural convection heat sink (not shown). Such heat sink can have no electrical fans attached and can have large spacings between its fins.

In some embodiments, the contemplated light fixture 100 does not have any heat sinks, that is, any heat sink having fins.

### Controls and Power Supply

Referring now to FIGS. 8 and 10, as discussed above, there can be one or more power supplies 130 disposed within the housing 102. These power supplies 130 can be connected to a power source, such as a 12V power source, via the power connector 108 (see FIG. 3). In some embodiments, the number of power supplies 130 directly correlates with the number of LED flexible printed circuits 120. The power supplies 130 can be disposed directly above the cooling tube 150. There can be a power board 132 electrically connected to the power supply 130. In some embodiments, the power board 132 can be electrically connected to the buss flexible printed circuit 140. The power board 132 may contain the relays, fuses, 12V and 5V converters, and a Wi-Fi microcontroller. Power board 132 can have the ability to auto-turn off the LEDs 121, 122, 124, if the temperature is too high on the body of the cooling tube 150.

In one aspect of the embodiments disclosed herein, there can be a buss flexible printed circuit (buss FPC) 140 disposed within the housing 102. In some embodiments, the buss flexible printed circuit 140 may be elongated and have a length that substantially correlates with the total length of the power supplies 130. For example, in the embodiment shown in FIG. 8, the buss flexible printed circuit 140 is sufficiently long to line the bottom of all three power supplies 130. The buss flexible printed circuit 140 may contain temperature sensors in multiple locations. In some embodiments, the buss flexible printed circuit 140 may contain temperature sensors in nine locations; in some embodiments, 18 locations; in some embodiments, at least one location for each power supply 130.

In other embodiments, the buss flexible printed circuit 140 may contain elements to control the water misting control valve. In some embodiments, the buss flexible printed circuit 140 may contain a gate and source lines for MOSFETs, as well as the ground, live, and neutral wires.

In one aspect of the disclosure, the misting schedule may be remotely changed, controlled, or auto adjusted using sensors. In another aspect of the disclosure, the lighting schedule may be remotely changed, controlled, or auto adjusted using sensors. In yet another aspect of the disclosure, the ratio of light output from high-power white LED 121:light output from Red LED 122:light output from UV LED 124 may be remotely changed, controlled, or auto adjusted using sensors. In still yet another aspect of the disclosure, the ratio of light output from UVA LEDs within the UV LEDs 124:light output from UVB LEDs within the UV LEDs 124:light output from UVC LEDs within the UV LEDs 124, may be remotely changed, controlled, or auto adjusted using sensors.

Various types of sensors and feedback control may be implemented. In one embodiment, the sensors and/or feedback control can alert the user to adjust lighting, UV levels, ratio of red/white/UVA/UVB/UVC, timing, and/or misting. Alternatively, the sensors and/or feedback control allows the lighting system to automatically adjust lighting, UV levels, ratio of red/white/UVA/UVB/UVC, timing, and misting. For example, such lighting system may be implemented in a residence and the lighting system can automatically inactivate UVA and/or UVB when a motion sensor detects the presence of people or pets. In this way, people or pet would not be unnecessarily over-exposed to UV light. In another example, the contemplated LED light fixture 100 can dim UVA and/or UVB to a desirable level (e.g., from 20 to 15, or down to a pre-selected level or a level determined in real time based on data and other feedback) and turns it off after a desirable amount of time when the motion sensor detects presence of people or pets. In this way, people or pets in the residence would not be exposed to the UV light for longer than a pre-determined amount of time. In yet another example, the contemplated LED light fixture 100 may receive data or signals from an outside source in real time (e.g., from a government agency, from the World Health Organization, from a central management office, from a contact-tracing applet/software/database, from a weather forecast agency) via a network (e.g., the Internet) so that the contemplated LED light fixture 100 can automatically adjust itself base these data or signals. In this way, the contemplated LED light fixture 100 can interact with the outside source and adjust itself based on perceived local threat. The contemplated LED light fixture 100 may also send data to an outside source. Sensors may also detect and help the contemplated LED light fixture 100 determine how fast people are moving through a space (e.g., a membership warehouse, a grocery store, an indoor merchandise retailer) and then adjust UV exposure time and level accordingly, so that shoppers are not unnecessarily exposed to UV for too long. In another aspect of the disclosure, the contemplated LED light fixture 100 may also automatically determine the preferred UV exposure time for people in the space based on locality. This can be done by automatically collect data made available to the contemplated LED light fixture 100 from any of the outside sources mentioned above. For example, shoppers in high latitude may be determined by the contemplated LED light fixture 100 to have a different tolerance of UV when compared with shoppers in lower latitude or near the equator.

While not wishing to be bound by any theory or hypothesis, the inventor has discovered that a highly dense LED array can easily match the PPFD (photosynthetic photo flux density) of any commercially available plant growth light. Such configuration of highly dense LED array in combination with the herein disclosed features can provide a LED light fixture with a much lower operating cost with drastically better cooling efficiency.

### Test 1:

Power at 650 watts, using 174 LEDs, having a 56 mm by 280 mm footprint, the contemplated light fixture 100 achieved 3-5 times the output for PPFD (photosynthetic photo flux density) than a GAVITA PRO E Series 600e SE 120/240V. PPFD is defined as a measure of the number of photons in the 400-700 nm range of the visible light spectrum (photosynthetic active radiation or PAR) that fall on a square meter of target area per second.

### Test 2:

Power at 650 watts, measured on a 5 foot by 5 foot grid, 36 inches from the LED light to the sensor, using triple the number of LEDs, densely arranged, with UV LEDs pulsating.

Thus, specific embodiments and applications of LED light fixture have been disclosed. It should be apparent, however, to those skilled in the art that many more modifications besides those already described are possible without departing from the disclosed concepts herein. The disclosed embodiments, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

What is claimed is:

1. A LED (light-emitting diode) light fixture comprising:
a LED flexible printed circuit, wherein said LED flexible printed circuit has a first array of high-power white LEDs to produce a first output of light;
the LED flexible printed circuit having a second array of red LEDs to produce a second output of light;
the LED flexible printed circuit having a third array of UV LEDs to produce a third output of light;
wherein the third array of UV LEDs includes at least one of UVA LEDs, UVB LEDs, and UVC LEDs.

2. The LED light fixture as recited in claim 1, wherein the third output of light is about 5% of a total output of light.

3. The LED light fixture as recited in claim 1, wherein the third output of light is between 2% and 8% of a total output of light.

4. The LED light fixture as recited in claim 2 wherein an output of light of UVA LEDs and UVB combined is about 5% of the third output of light.

5. The LED light fixture as recited in claim 4 wherein said total output of light is defined as a luminous power flux.

6. The LED light fixture as recited in claim 1, wherein a ratio of said first output of light:said second output of light:said third output of light is user-adjustable to achieve a germicidal effect.

7. The LED light fixture as recited in claim 1, wherein based on an information received about an environment, the LED light fixture automatically adjusts at least one of the following:
a) a ratio of said first output of light:said second output of light:said third output of light;
b) a time schedule to turn on and off the LEDs;
c) a spraying schedule.

8. The LED light fixture as recited in claim 1, wherein said LED light fixture comprises a liquid cooling assembly capable of containing a liquid, wherein the LED flexible printed circuit is coupled to the liquid cooling assembly.

9. The LED light fixture as recited in claim 8 wherein the LED flexible printed circuit is directly attached to the liquid cooling assembly.

10. The LED light fixture as recited in claim 9 further comprising a power supply disposed on the liquid cooling assembly, opposite to said first, second, and third arrays.

11. The LED light fixture as recited in claim 10, wherein LED flexible printed circuit has a portion that wraps around to a top side of the liquid cooling assembly, said portion having at least one pin to electrically connect to a contact of the power supply.

12. The LED light fixture as recited in claim 8, further comprising a second liked LED flexible printed circuit coupled to the liquid cooling assembly.

13. The LED light fixture as recited in claim 8 further comprising at least one nozzle fluidly connected to the liquid cooling assembly to spray said liquid.

14. The LED light fixture as recited in claim 13, wherein said liquid is a disinfectant.

15. The LED light fixture as recited in claim 8 further comprising at least one radiator fluidly connected to the liquid cooling assembly via a tube, wherein the at least one radiator is physically located away from the LED light fixture, and the at least one radiator is located:
A) indoor,
B) outdoor, or
C) both.

16. The LED light fixture as recited in claim 1 further comprising at least one electric fan coupled to a solid-to-air heat exchanger having fins, said electric fan drives an air flow, and wherein the LED flexible printed circuit is coupled to the solid-to-air heat exchanger.

17. A method to sterilize surfaces and aerosols indoors for viruses, bacteria, or mold, the method comprising:
activating a first array of high-power white LEDs in a LED light fixture to produce an output of light;
activating a second array of red LEDs in the LED light fixture to produce an output of light;
activating a third array of LEDs in the LED light fixture to produce an output of light, wherein the third array of LEDs having at least one of UVA LEDs, UVB LEDs, and UVC LEDs;
wherein said first array, second array, and third array are disposed on a LED flexible printed circuit;
adjusting a ratio of outputs of said first array:said output of said second array:said output of third array, to achieve a total output of light, which is defined as a luminous power flux.

18. The method as recited in claim 17, wherein the output of light of the third array of LEDs is about 5% of the total output of light.

19. The method as recited in claim 18, wherein an output of light of UVA LEDs and UVB LEDs combined is about 5% of the output of light of the third array of LEDs.

20. The method as recited in claim 17, cooling the LED light fixture by flowing a liquid through a cooling assembly which directly or directly makes physically contact with the LED flexible printed circuit.
